(19) United States
(12) Patent Application Publication
(10) Pub. No.: US 2015/0065875 A1
(43) Pub. Date: Mar. 5, 2015

(54) NAVIGATION ATTACHMENT AND UTILIZATION PROCEDURE

(71) Applicant: SurgicEye GmbH, Munchen (DE)

(72) Inventor: Michael FRIEBE, Recklinghausen (DE)

(21) Appl. No.: 14/473,674

(22) Filed: Aug. 29, 2014

(30) Foreign Application Priority Data
Aug. 30, 2013 (DE) 1020131094864

(51) Int. Cl. A61B 19/00; A61B 8/00; A61B 6/02

(52) U.S. Cl. CPC A61B 19/2203; A61B 6/022; A61B 8/54; A61B 19/20; A61B 2019/2207
USPC 600/436; 600/459; 600/439; 606/130

(57) ABSTRACT

A navigation attachment for a manually guided instrument which is designated for the surgical and/or therapeutic treatment of a body and/or for implementation of a diagnostic procedure on the body is provided. The navigation attachment includes a fastener adapted to be fixedly connected with the instrument; at least one image pickup unit, including a device to determine a distance between the navigation attachment and a surface of the body; wherein at least three distances are usable to represent a position (x, y, z, t) of the instrument and/or a front section of the instrument in relation to the body or to a component of the body. Further, a navigation procedure is provided.

### RELATED APPLICATIONS

This application claims priority to Germany Application Serial Number 102013109486.4 filed Aug30, 2013, herein incorporated by reference.

This disclosure relates to medical technical devices and utilization procedures. It specifically relates to devices and procedures for the precise manipulation of a manually or robotically guided device, such as, a surgical instrument, an ultrasound transducer, or of another diagnostic system.

### TECHNICAL BACKGROUND

The generation of high-quality images and/or the tracking of operational instruments, e.g. on a monitor, is of great importance for a wide range of applications. Particularly in medicine, where patient health is at issue, the best possible image generation and/or positioning for instruments and probes is required, for example as a basis for operations on patients.

In particular, tracking of handheld probes used as typical diagnostic devices is well-known, particularly during surgical interventions, as well as tracking systems which determine the position and orientation of operational instruments and imaging devices.

The exact positioning of instruments and probes is often made more difficult due to the complex makeup of the assistive technologies, in particular of hooks, clamps, clips, fasteners, tubes, catheters, capillary tubes, probes, electrodes, or other instruments or assistive technologies, as well as due to the presence of the hands and arms of the personnel involved in the imaging/therapy, since direct visual contact with the utilization area can be made more difficult or cut off. Likewise, problems can arise if an instrument or probe is temporarily not in the field of vision of an externally installed camera system and, so, at least temporarily, cannot be controlled or tracked. In addition, electromagnetic image disruptions can occur due to individual system component interference. Given these and other problems, the task of the present invention is to make a better device and a procedure available.

### SUMMARY

In view of the above, a navigation attachment and a navigation procedure in accordance with embodiments proposed.

An embodiment of the invention relates to a navigation attachment for a manually guided instrument which is designated for the surgical and/or therapeutic treatment of a body and/or for carrying out a diagnostic procedure on a body. The navigation attachment includes a fastener adapted to be fixedly connected with the instrument; at least one image pickup unit, including a device to determine a distance between the navigation attachment and a surface of the body; wherein at least three distances are usable to represent a position (x, y, z, t) of the instrument and/or a front section of the instrument in relation to the body or to a component of the body.

Another embodiment relates to a navigation procedure for a manually or robotically guided medical instrument. The navigation procedure comprises providing a navigation attachment, which includes a fastener adapted to be fixedly connected with the instrument; at least one image pickup unit, including a device to determine a distance between the navigation attachment and a surface of the body; wherein at least three distances are usable to represent a position (x, y, z, t) of the instrument and/or a front section of the instrument in relation to the body or to a component of the body; rigidly connecting the navigation attachment to the medical instrument; determining at least three distances between the navigation attachment and a body in relation to which the medical instrument is to be placed in a predetermined position; determining based on the at least three distances, position data (x, y, z, t) related to the body and/or one of its components for at least a front section of the medical instrument; representation; by an image rendering unit, of at least the front section in an image of the body and/or one of its components.

Additional features, aspects, and details, which can be combined with the embodiments described here, will be disclosed in the dependent claims, the description, and the drawings.

### BRIEF DESCRIPTION OF THE DRAWINGS

The attached drawings show embodiments of the proposed navigation attachment and serve, together with the description, to explain the principles of the invention. The elements of the drawings are shown in relation to each other and are not necessarily drawn to scale. Identical reference numbers identify corresponding similar elements.

FIG. 1A—a schematic overall view of the navigation attachment placed on the back section of a medical instrument;

FIG. 1B—example embodiments of an attachment on an instrument without direct contact with the body;

FIG. 1C—example embodiments of a navigation attachment on an instrument in contact with the body;

FIG. 1D—a schematic frontal view of an example embodiment with 4 camera systems;

FIG. 2A shows a navigation attachment in use in example embodiments together with a medical instrument on a body with markings;

FIG. 2B shows a navigation attachment in use in further example embodiments together with a medical instrument on a body with markings.

### DETAILED DESCRIPTION

Various embodiments concern a cap fitted with a camera assembly which is attached to a side of the instrument directed away from the body, typically to an instrument grip, which is *locked with the instrument and which allows a pictorial representation of the instrument in a visualization of the body or of a body section generated in another fashion, without impairing the otherwise generated visualization, for example via electromagnetic image disruptions. The cap, which is permanently attached to the grip of the instrument in question in some embodiments, supplies the instrument's current spatial coordinates via the registration of a distance from and an angle to at least one of the markers placed on the body, in interaction with an offsetting unit and an output unit, as well as with a related time unit (4D), in particular with respect to its front section, in relation to the body with the marker, and/or allows a schematic representation of the instrument in an independently created image of the body or of the body section in question. This allows an operator—even in the absence of direct visual contact with the front end if the instrument—to manipulate the instrument precisely using the independently generated image of the body or body section. The navigation device can, in addition to the diagnostic overlapping of multiple procedures (e.g. ultrasound and gamma camera), also be used to advantage in the area of minimally invasive surgery and image-guided interventions/therapies.

In some embodiments, the navigation attachment is adapted to visually controlled—via an image rendering unit—manipulation and representation of the instrument on or in the body of a patient.

In some embodiments, a navigation attachment is proposed that can be secured or locked to a manually or robotically guided instrument designated for surgical or therapeutic interventions and/or the implementation of diagnostic procedures on a body. Here “body” is understood as the body of a human or animal organism, or, in the case of non-medical diagnostic/therapeutic applications, as the respective non-human body to be handled (for example, when inspecting the material of components/systems). The navigation attachment can be rigidly fixed to the instrument and also has at least one image pickup device, which includes a device to determine the distance and, optionally, the angle between the navigation attachment and a surface of the body and/or a body axis. Based on a determination of at least three distances between the navigation attachment and the surface of the body, one can determine a position (x, y, z) of the instrument at the given point in time and/or a position of a front section of the instrument in relation to the body or a component of it at the given point in time t. The same is also true if there are at least three different markers at a point on the body which can be recognized by the navigation attachment in one position.

An advantage provided by some embodiments is that the position determined can be rendered by means of an output unit, such that operator can more easily guide the instrument as its position is shown—e.g. on a previously recorded data image—or due to the fact that the recorded instrument data is superimposed on a data image, or the fact that the recorded instrument data allows generation of an independent 3D image or precise positioning of the instrument in a previously recorded data image, to which the navigation data are referenced. There are additional advantages, especially if the user of the instrument, i.e. the operator, already sees the body or body section—which is being or will be subjected to a treatment or a more detailed diagnosis with the instrument—in the form of a pictorial representation but the instrument in question does not correctly render this representation correctly or if its correct rendering with the same technique would impair the current image quality. For example, the proposed navigation attachment allows the instrument to be represented without further loading of the body with additional contrast media or other marking aids, and without electromagnetic impairment of the existing image quality. Based on the respective current navigation attachment's positions—which, if need be, can be updated at very short time intervals—a pictorial representation of the instrument or its front section is imported into an image generated in accordance with another representation principle, superimposed upon it, or made visible within it. This has the great advantage of allowing the operator to navigate the instrument precisely in the respective body, body section, or tissue.

In some embodiments, a navigation attachment is proposed with a recess designed to be reversibly and—at least in sections—form-fittingly combined with a back section, for example with a grip-end, of the instrument, with at least one image pickup unit being placed next to the recess. For example, the recess can be designed such that, in a specific orientation, it locks to the instrument, i.e. such that the attachment can be combined with 30 the instrument form-fittingly in sections.

An advantage of these features is that one and the same navigation unit can be used universally for various instruments. When instrument-specific parameters (length, any curvatures, etc.) are known, positions determined can be calibrated and/or the position of a front section of the instrument can be referenced with the aid of the navigation attachment.

In some embodiments, a navigation attachment is proposed with three image pickup units, each of which is adapted to record a distance to at least one marker on the surface of the body and, optionally, to determine the axial rotation with respect to the marker, if the marker is in the detection area of the image pickup unit.

The advantage lies in being able to standardize an allocation algorithm with the distances received by means of standardized markers. This increases the reliability of the calculated positions and/or spatial coordinates.

In some embodiments, a navigation attachment is proposed, adapted to transmit values determined to an evaluation unit connected to an output unit, with the evaluation unit being set up to allow a representation of the instrument and/or at least its front section. The image received can also be embedded in an existing image of the body or a component of it.

The advantages include improved capability to conduct inspections and treatments e.g. for diagnostic and/or therapeutic purposes, in particular when the imaging is done with an image rendering unit.

In some embodiments a navigation attachment is proposed in which the front end of the instrument includes an ionizing radiation source or an ultrasonic probe. In particular, the manipulation of ionizing radiation sources, for example for an intended therapeutic effect, should be done with precise dosages. This presupposes precise placement of the radiation source. Thus, an advantage of these embodiments is that they allow precise surface, interstitial, or interventional brachytherapy.

In some embodiments, a navigation procedure is proposed, for a manually or robotically guided medical instrument. The navigation procedure includes the steps:—furnishing a navigation attachment in accordance with at least one of the embodiments described above;—fixed connection of the navigation attachment with the medical instrument;—determination of at least three distances between the navigation attachment and a body in relation to which the medical instrument is to be placed in a predetermined position;—determination, based on at least three distances, of position data related to the body and/or one of its components for at least the front section of the instrument;—representation by an image rendering unit of at least the front section in an image of the body and/or its components.

The advantages of the procedure described lie in the possibility of greater precision with limited direct visual control during an intervention or a local treatment such as irradiation.

In some embodiments, the navigation procedure also includes the manual or robotic correction of a position of the front section of the instrument using current position data (x, y, z, t), at least of a front section.

Advantages include, for example, the possibility of rapid correction of the position of the instrument in or on the body and the avoidance of unintended effects. In particular, the use of current position data (x, y, z) at the point in time t can include the visual observation of an image of the instrument, for example on a monitor or a projection screen, in the image of the body or body section to be treated.

In some embodiments, the navigation procedure also includes the emission of a signal as soon as a previously determined minimum distance is reached between the front section of the medical instrument and a definable area in the image and/or a definable structure in the image and/or this minimum distance is undershot.

The advantages of these embodiments include constant control and the ability to maintain a minimum distance from the structures to be excluded from the treatment or from direct contact with the instrument. For example, unintended damage to or destruction of tissue next to an operating field can be avoided.

The above-described embodiments can be combined with each other at will. Several embodiments can be chosen and combined with each other. Likewise, all the embodiments can also be combined with each other, eventually plus or minus certain or several specific features.

In general, the navigation attachment embodiments described make it easier for an operator to accurately place an instrument and/or probe and/or to represent the instrument and/or probe, or its front section, in its true position in an image of a body section.

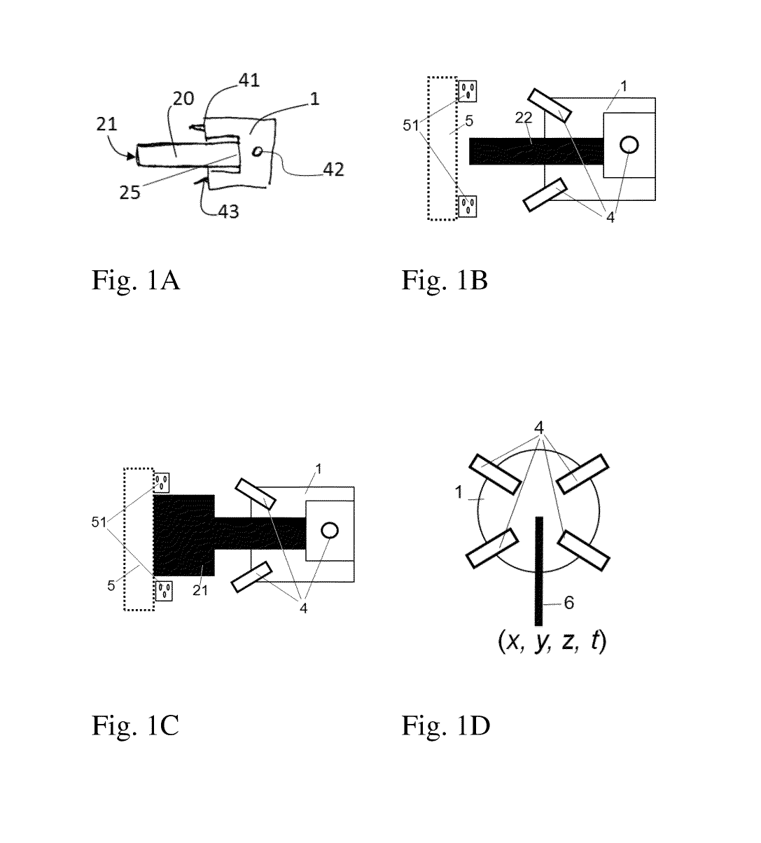

In FIG. 1 in particular, the navigation attachment 1 is shown with a recess enclosing the back end 25 of an instrument 20. The navigation attachment 1 embodiment shown in FIG. 1A includes a navigation attachment 1 with three distance-measurement devices and/or three camera systems 41, 42 und 43. These can, for example, be image pickup units (digital camera modules) with an auto-focus function, whereby, based on at least three determined values, a current position (x, y, z, t) of the navigation attachment 1 can be determined in relation to the body 5 and/or marker 51 on the surface of body 5. This feature is based on the principle of triangulation. Specifically, object distances can be determined from image distances based on the lens and/or distance equation. If this is done for three different marker positions 51 and/or different marker/image pickup unit pairs, the result is a unique position of the navigation attachment 1 in the space above body 5 (the purely theoretical position lying behind or in the body is discarded). The corresponding position or spatial coordinates can be determined with a suitable circuit in navigation attachment 1 itself Likewise, the processing of discrete distances can be done in a separate control and processing unit not shown in the figures. One advantage of this is that the control and processing unit can also take over the transmission of corresponding signals to represent the instrument or its front portion in an existing image of the body or a corresponding section of the body or an operating or treatment field.

A different number of image pickup units 4 can be provided for different applications. For example, in the schematic drawing (FIG. 1A) three camera systems 41, 42 und 43 are arranged such that each can record a marker on the surface of the body. Using the spatial coordinates determined, and if the (instrument-specific) distance and orientation of the navigation attachment with respect to a front end 21 of the instrument 20 and/or 22 are known, a known position of the instrument 20, 22 in relation to the body 5 or the surface of body 5 can be determined.

FIG. 1B shows navigation attachment 1 employed with an instrument 22 that is not used in direct contact with the body of a patient 5 or the surface of body 5. Such an instrument 22 can, for example, be a gamma camera or a gamma probe.

FIG. 1C shows navigation attachment 1 employed with an instrument 21 that is used in direct physical contact with the body of a patient 5 or the surface of body 5. Such an instrument can, for example, be a diagnostic system such as an ultrasonic probe, which, via a coupling, sends ultrasonic signals into the body and captures the return signals, which it then again transduces into electrical signals (so-called ultrasonic transducers).

The distances determined or the corresponding raw data can be transmitted via a cable 6. However, the information (e.g. geometry, navigation, and/or time data) can also be transmitted wirelessly from the navigation attachment to a control and processing unit, for example via Bluetooth. A frontal view of a corresponding navigation attachment is shown schematically in FIG. 1D.

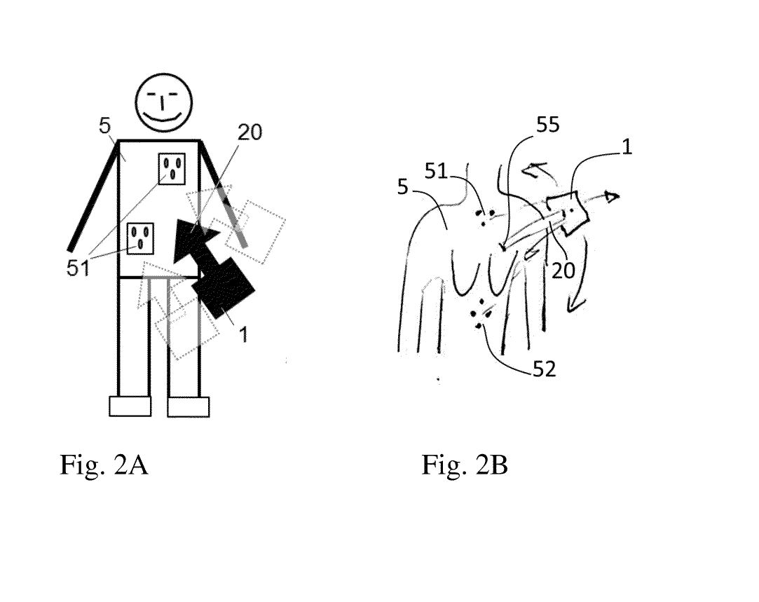

FIGS. 2A and 2B show different intermediate positions (represented as dotted lines or arrows) of instrument 20 with a navigation attachment 1 in relation to markers 51 and 52 fixed to body 5. As can be seen, the markers can be similar 51 (cf. FIG. 2A) or different in different positions of the body 51, 52 (cf. FIG. 2B). Markers suitable as references for position information, e.g. as a patch, include infrared markers such as infrared transmitters or reflectors; a barcode; a QR code; or a data matrix code. Likewise the position of an opening 55 in the surface of the body, into which the instrument 20 is introduced, may be known. The static markers 51, 52 allow the position of the navigation attachment 1 above the body 5 to be determined. Similarly, body markers (such as the nose, an ear, an eyebrow, etc.) can be used as additional reference markers. The advantages of a precise positioning include the exact positioning of a radiation source at the precisely known—and perhaps synchronously with another image-producing procedure—depicted place of a malignant tissue change. The standard electromagnetic tracking procedures employed cover only a small portion of the body. Thus, the maximal distance allowed in a commercial tracking system (NDI) is, for example, approximately 40 cm from the data cube. The dimensions of the patient's body sometimes lie outside the measuring volume characterized by the tracking system. Likewise, metallic components can affect measurement accuracy. The cameras of common optical tracking procedures are typically placed very far from the patient and therefore often no longer even allow a direct view of the system “to be tracked.” With a camera attachment such as that described here in navigation attachment 1, the disadvantages described here as examples can be avoided, and additional markers, including body markers such as the nose, an ear, or an eyebrow, can be used as additional reference markers.

The disadvantages of the common devices and procedures described above can be overcome by means of the described embodiment and the examples given. The complex integration of electronic circuits and component assemblies that can be realized with established technologies allows the described navigation attachment to be compactly built. For example, miniaturized camera modules, which are common in the telecommunications area (terminals), can be employed with the described navigation attachment. Likewise, adapted circuits (e.g. ASICs) and components of micro-electromechanical systems (MEMS) such as position sensors can be used. Thus, in accordance with another embodiment, ultrasonic distance sensors could be arranged on the base component instead of the image pickup units or in combination with them. Likewise, infrared sensors, electronic (including capacitive and inductive) distance sensors, wireless modules, or suitable opto-electrical components assemblies can be combined among and/or with each other for distance measurement purposes in order to allow an image-generation procedure independent of the respective primary imaging system or one which does not disrupt that system.

The above concerns various embodiments of the invention, however, other and additional embodiments would occur to those skilled in the art without departing from the scope and the spirit of the present invention as specified by the following claims.

What is claimed is:

1. A navigation attachment for a manually guided instrument which is designated for the surgical and/or therapeutic treatment of a body and/or for carrying out a diagnostic procedure on a body, including:
a fastener adapted to be fixedly connected with the instrument;
at least one image pickup unit, including a device to determine a distance between the navigation attachment and a surface of the body;
wherein at least three distances are usable to represent a position (x, y, z, t) of the instrument and/or a front section of the instrument in relation to the body or to a component of the body.

2. The navigation attachment according to claim 1 wherein the navigation attachment includes a recess designed to be reversibly and—at least in sections—form-fittingly combined with a back section of the instrument, with the at least one image pickup unit being placed next to the recess.

3. The navigation attachment of claim 1, including one to six image pickup units adapted to determine a distance to at least one marker on the surface of the body and, optionally, to determine an axial rotation with respect to the marker if the marker is in the detection area of the image pickup unit.

4. The navigation attachment of claim 1 adapted to transmit determined distances to an evaluation unit connected to an image rendering unit, with the evaluation unit being set up to allow a representation by the image rendering unit of the instrument and/or its front section and/or to enable the embedding of this representation in an existing image of the body or an existing image of a body component.

5. The navigation attachment of claim 1, wherein the front section includes a radiation source for ionizing radiation or an ultrasonic probe.

6. Navigation procedure for a manually or robotically guided medical instrument, comprising:
providing a navigation attachment according to claim 1;
rigidly connecting the navigation attachment to the medical instrument;
determining at least three distances between the navigation attachment and a body in relation to which the medical instrument is to be placed in a predetermined position;
determining, based on the at least three distances, position data (x, y, z, t) related to the body and/or one of its components for at least a front section of the medical instrument;
representation by an image rendering unit, of at least the front section in an image of the body and/or one of its components.

7. The navigation procedure of claim 6 further comprising:
manual or robotic correction of a position of the front section with current position data (x, y, z, t) of at least the front section.

8. The navigation procedure of claim 6 further comprising:
emission of a signal, if the front section reaches and/or undershoots a pre-determinable minimum distance from a definable area in the image and/or a definable structure in the image.
